(19) United States
(12) Patent Application Publication
(10) Pub. No.: US 2024/0091499 A1
(43) Pub. Date: Mar. 21, 2024

(54) Protective Cap for Landmarking Medical Device

(71) Applicant: Bard Access Systems, Inc., Salt Lake City, UT (US)

(72) Inventors: Steffan Sowards, Salt Lake City, UT (US); William Robert McLaughlin, Bountiful, UT (US)

(21) Appl. No.: 18/369,682

(22) Filed: Sep. 18, 2023

Related U.S. Application Data
(60) Provisional application No. 63/408,054, filed on Sep. 19, 2022.

(51) Int. Cl. A61M 25/00

(52) U.S. Cl. CPC A61M 25/0082; A61M 2205/11

(57) ABSTRACT

An intravascular medical device assembly, including a tubular member or catheter defining a lumen, an elongate medical device disposed within the lumen, and a cap attached to and covering the distal end of the tubular member, where the cap is configured to prevent contamination and damage of the elongate medical device. The elongate medical device includes an optical fiber for shape sensing and magnetic elements for magnetic tracking of the device assembly during advancement along a patient vasculature. The cap is configured to form a fluid tight seal with the tubular member.

### PRIORITY

This application claims the benefit of priority to U.S. Provisional Application No. 63/408,054, filed Sep. 19, 2022, which is incorporated by reference in its entirety into this application.

### BACKGROUND

The tracking of medical devices, such as intravascular medical devices within a patient, provides for enhanced of accuracy of placement and reduced patient risk resulting from incorrect placement. One example is the placement of a central catheter within the superior vena cava. In some cases, a medical device having tracking capability may be disposed within a catheter lumen. Handling of the catheter prior to insertion may allow the distal portion of the medical device to be contaminated via the open distal end of the catheter.

Disclosed herein are devices and methods for protecting and preventing contamination when inserted within a catheter, thereby addressing the items described above.

### SUMMARY

Briefly summarized, disclosed herein is a medical device assembly, that according to some embodiments, includes (i) a tubular member defining a lumen extending along a length of the tubular member from a proximal end to a distal end, where the tubular member is configured for insertion into a patient, (ii) an elongate medical device disposed within the lumen such that a distal end of the elongate medical device is disposed adjacent the distal end of the tubular member, and (iii) a cap covering the distal end of the tubular member, where the cap is configured to prevent contamination and/or damage of the elongate medical device during use.

In some embodiments, the cap forms a fluid tight seal with the tubular member, and in some embodiments, the cap is configured to prevent microbial ingress into the lumen. In some embodiments, the cap is detachably coupled with the tubular member.

In some embodiments, the elongate medical device is configured to enable tracking of a location of the elongate medical device within the patient.

In some embodiments, the elongate medical device includes an optical fiber extending along a length of the elongate medical device. In some embodiments, the optical fiber is multicore optical fiber including a plurality of fiber optic gratings disposed along a length of the optical fiber, the fiber optic gratings configured to enable shape sensing of the elongate medical device.

In some embodiments, the multicore optical fiber includes (i) a number of illuminating optical fiber cores configured for propagating illuminating light distally along the elongate medical device and projecting the illuminating light away from the distal end of the elongate medical device; and (ii) a number of imaging optical fiber cores configured for propagating imaging light proximally along the elongate medical device, where the imaging light is received at the distal end of the elongate medical device in response to the illuminating light.

In some embodiments, the elongate medical device includes a number of magnetic elements disposed along at least a distal portion of the length of the elongate medical device, where the magnet elements are configured to enable magnetic tracking of a location the elongate medical device. In some embodiments, the number of magnetic elements define a magnetic signature for the medical device, where the magnetic signature is distinct from other magnetic signatures for other elongate medical devices.

In some embodiments, the catheter is an intravascular catheter, and in some embodiments, the elongate medical device is a stylet, a catheter, a needle, an obturator, or a sheath.

Also disclosed herein is a medical cap for a vascular catheter that, according to some embodiments, includes a cap body defining a proximal end and a distal end and a cavity extending into the cap body from the proximal end, where the cavity is configured to receive and couple with the catheter.

In some embodiments, the cap forms a fluid tight seal with the catheter, and in some embodiments, the cap is configured to prevent microbial ingress into the lumen.

In some embodiments, the cavity includes a tapered inlet, and, in some embodiments, a distal end of the cap includes a bullnose shape.

Also disclosed herein is a method of inserting a medical device into a patient that, according to some embodiments, includes providing an medical device assembly, where the medical device assembly includes (i) a tubular member defining a lumen extending along a length of the tubular member from a proximal end to a distal end, where the tubular member is configured for insertion into the patient; (ii) an elongate medical device disposed within the lumen such that a distal end of the elongate medical device is disposed adjacent the distal end of the tubular member; and (iii) a cap covering the distal end of the tubular member, where the cap is configured to prevent contamination and/or of the elongate medical device. The method further includes removing the cap from the tubular member and inserting the medical device assembly through a skin of the patient.

In some embodiments of the method, the cap forms a fluid tight seal with the tubular member, and in some embodiments, the cap is configured to prevent microbial ingress into the lumen.

In some embodiments, the method further includes advancing the medical device assembly along a vasculature of the patient. In some embodiments, the method further includes tracking the location of the elongate medical device during while advancing the medical device assembly.

In some embodiments of the method, the elongate medical device includes a multicore optical fiber including a plurality of fiber optic gratings disposed along a length of the optical fiber, where the fiber optic gratings are configured to enable shape sensing of the elongate medical device, and where tracking the location of the elongate medical device includes monitoring a shape of the elongate medical device depicted on a display of a fiber optic shape sensing system.

In some embodiments of the method, the multicore optical fiber includes (i) a number of illuminating optical fiber cores configured for propagating illuminating light distally along the elongate medical device and projecting the illuminating light distally away from the distal end of the elongate medical device, and (ii) a number of imaging optical fiber cores configured for propagating imaging light proximally along the elongate medical device.

In some embodiments, the elongate medical device includes a number of magnetic elements disposed along at least a distal portion of the elongate medical device, and tracking the location of the elongate medical device includes tracking the location of the number of magnetic elements with respect to a number of magnetometers of a magnetic tracking system.

In some embodiments of the method, the number of magnetic elements define a magnetic signature of the elongate medical device, and in some embodiments of the method, where the magnetic signature that is distinct from other magnetic signatures of other elongate medical devices.

In some embodiments of the method, the tubular device is an intravascular catheter or a sheath, and in some embodiments of the method, the elongate medical device is a stylet, a catheter, a needle, or an obturator.

These and other features of the concepts provided herein will become more apparent to those of skill in the art in view of the accompanying drawings and following description, which disclose particular embodiments of such concepts in greater detail.

### DRAWINGS

Embodiments of the disclosure are illustrated by way of example and not by way of limitation in the figures of the accompanying drawings, in which like references indicate similar elements and in which:

FIG. 1 is an illustration of a medical device assembly, in accordance with some embodiments;

FIG. 2 is an illustration of the medical device assembly of FIG. 1 in use with a patient, in accordance with some embodiments; and

FIG. 3 illustrates a flow chart of a method of inserting the medical device assembly of FIG. 1 into a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

The phrases “connected to,” “coupled with,” and “in communication with” refer to any form of interaction between two or more entities, including but not limited to mechanical, and fluid interaction. Two components may be coupled with each other even though they are not in direct contact with each other. For example, two components may be coupled to each other through an intermediate component.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations may be made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

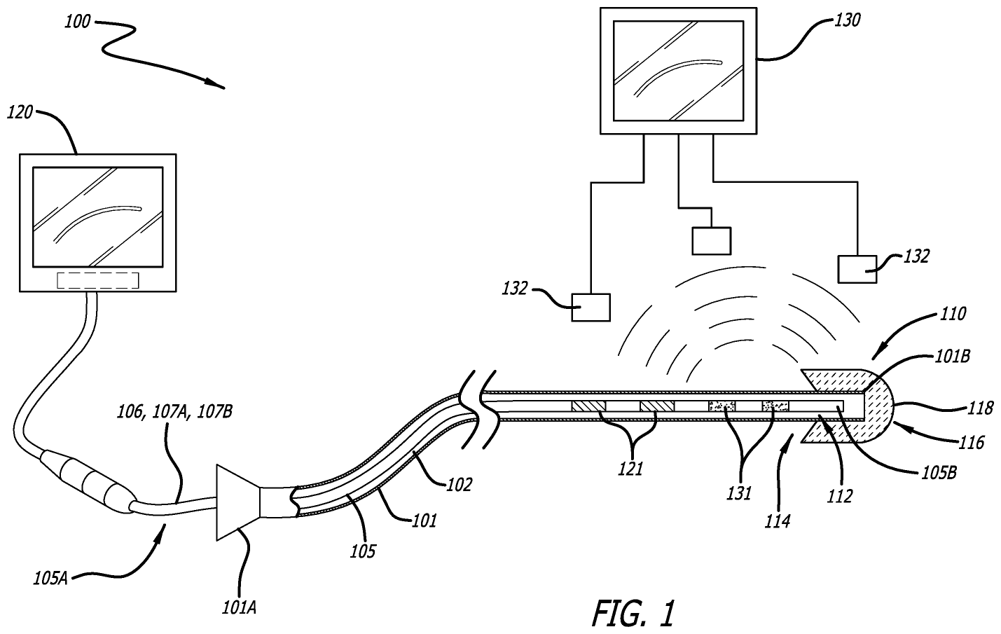

FIG. 1 illustrates a medical device assembly 100 that is generally enabled to facilitate landmarking (i.e., mapping or tracking the location of) the medical device assembly 100 during use. In the illustrated embodiment, the medical device assembly 100 is configured to enable tracking via at least one of fiber optic shape sensing or magnetic tracking of magnetic elements. As such, in some use cases, the location of the medical device assembly 100 may be tracked via a shape sensing system 120 coupled with optical fiber of the medical device 100. In other use cases, the location of the medical device assembly 100 may be tracked via a magnetic tracking of the magnetic elements coupled with the medical device 100 via a magnetic tracking system 130 having a number of magnetometers 132. In still other use cases, the medical device 100 may be tracked the shape sensing system 120 in combination with the magnetic tracking system 130.

The medical device 100 includes tubular member 101. The tubular member 101 defines a lumen 102 extending along a length of the tubular member 101 between a proximal end 101A and a distal end 101B. The tubular member 101 is configured for insertion into a patient, including advancement along a vasculature of the patient. In some embodiments, the tubular member 101 may be a catheter, such as a central venous catheter or a peripherally inserted central catheter, for example. As such, the tubular member 101 may be formed of a material consistent vascular catheters current available. In other embodiments, the tubular member 101 may be a sheath.

The medical device assembly 100 includes elongate medical device 105 extending along the length of the tubular member 101. In some embodiments, a distal end 105B of the elongate medical device 105 is disposed adjacent the distal end 101B of the tubular member 101. A proximal end 105A of the elongate medical device 105 may extend proximally away from the proximal end 101A of the tubular member 101. In the illustrated embodiment, the elongate medical device 105 is disposed within the lumen 102 of the tubular member 101. In some embodiments, the elongate medical device 105 may be slidably disposed within the lumen 102 of the tubular member 101. As such during use, the elongate medical device 105 may be inserted into and/or extracted from the tubular member 101. In some embodiments, the elongate medical device 105 may be a vascular device, such as a stylet, a catheter, a needle, a guidewire, an obturator, or a sheath, for example.

In some embodiments, the elongate medical device 105 includes an optical fiber 106 extending along the length of the elongate medical device 105. The optical fiber 106 is multicore optical fiber including a plurality (e.g., 10, 50, 100 or more) of fiber optic gratings 121 (e.g., fiber optic Bragg gratings) disposed along the length of the optical fiber 106, where the fiber optic gratings 121 are configured to enable shape sensing of the elongate medical device 105 by the shape sensing system 120.

In some embodiments, the optical fiber 106 includes a number of illuminating optical fiber cores 107A configured for propagating illuminating light distally along the elongate medical device 105 and projecting the illuminating light away from the distal end 10B of the elongate medical device 105. In such embodiments, the optical fiber 106 further includes a number of imaging optical fiber cores 107B configured for propagating imaging light proximally along the elongate medical device 105, where the imaging light received at the distal end 105B of the elongate medical device 105 is in response to the illuminating light.

In the illustrated embodiment, the elongate medical device 105 is configured to enable tracking or mapping of the location of the elongate medical device 105 during use, such as during advancement along a vasculature of the patient, for example. The elongate medical device 105 includes a number (e.g., 2, 3, 4, or more) of magnetic elements 131 disposed along at least a distal portion of the elongate medical device 105. The magnetic elements 131 may passive magnets or magnetized ferrous elements of the elongate medical device 105. The magnetic elements 131 may include dipoles having a length, an orientation, and a spacing between adjacent dipoles. In some embodiments, the length and orientation of the dipoles combined with the spacing between adjacent dipoles may define a magnetic signature of the elongate medical device 105, where the magnetic signature may be distinct from other magnetic signature of other elongate medical devices. The magnetic elements 131 define a magnetic field, e.g., a combination of multiple magnetic fields individually defined by each of the magnetic elements 131. The magnetic field enables magnetic tracking of at least the distal portion of the elongate medical device 105 by the magnetic tracking system 130.

The medical device assembly further includes a cap 110 configured to attach to and sealably cover the distal end 101B of the tubular member 101. The cap 110 is generally configured to prevent contamination and/or damage of the elongate medical device 105. The cap 110 may form a fluid tight seal with the distal end 101B of the tubular member 101. In some instances, the elongate medical device 105 or a distal portion thereof, may become contaminated during handling before insertion. As such, the cap 110 may prevent contamination that may, in some instances, alter the function of the elongate medical device 105. The cap 110 may also be configured to prevent physical damage to the distal end 105B of the elongate medical device 105. The cap 110 may also be configured to prevent microbial ingress into the lumen 102 to maintain a sterility of the elongate medical device 105 and other interior portions or surfaces of the tubular member 101.

The cap 110 may be elongate in shape having a proximal end and a distal end. The cap 110 includes a cavity 112 extending distally inward from a proximal of the cap 110. The cavity 112 is configured to receive the distal end 101B of the tubular member 101. The cavity 112 is configured to attach the cap 110 to the tubular member 110, such that the cap 110 remains attached until the cap 110 is intentionally removed by the clinician. In some embodiments, the cavity 112 may form an interference fit with the tubular member 101. In some embodiments, the cap 110 may be attached to the tubular member 101 via separable adhesive. In some embodiments, the cap 110 may be pre-attached to the tubular device 101 during manufacture of the tubular device 101. In other embodiments, the cap 110 may attached the tubular member 101 by the clinician. The cap 110 may be formed of a material similar to the catheter materials. In some embodiments, the cap 110 may be formed of an elastomeric material, such as silicone or EPDM, for example.

The cap 110 may be configured (e.g., shaped) to enhance handling of the cap 110. In some embodiments, a distal end of the cap 110 may include a bullnose shape 116, where the smooth contours of the bullnose shape 116 may inhibit the cap 110 from snagging or otherwise negatively engaging packaging materials for the medical device assembly 100 or other articles associated with using the medical device assembly 100. In some embodiments, the cavity 112 may include a tapered inlet 114 at the proximal end of the cap 110, where the tapered inlet 114 may help guide the tubular member 101 into the cavity 112 during attachment of the cap 110 to the tubular member 101.

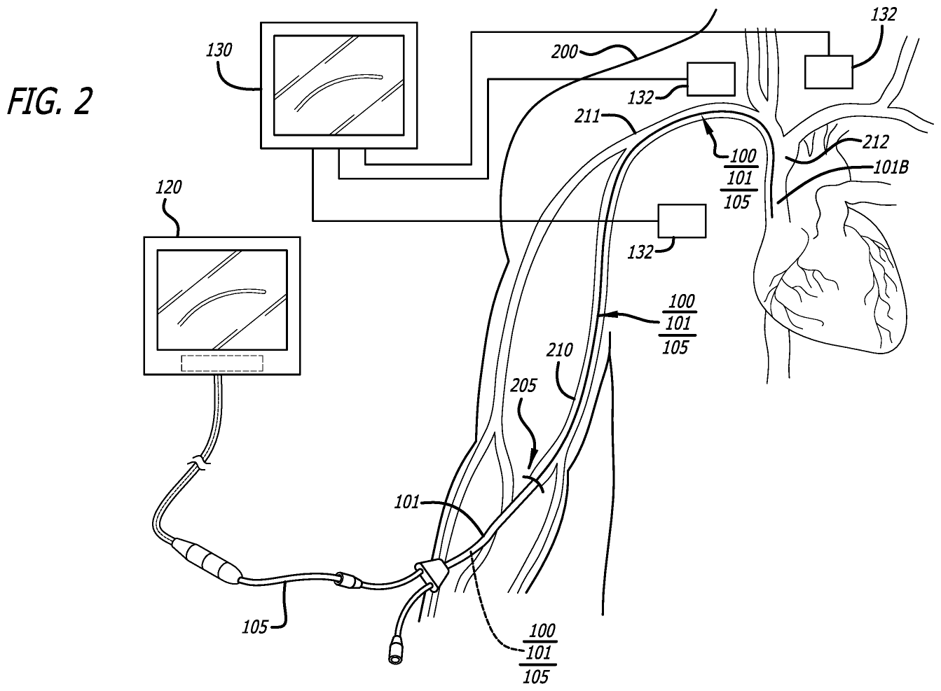

FIG. 2 illustrates an exemplary use case of the medical device assembly 100 with a patient 200. In the illustrated use case, the tubular member 101 of the medical device assembly 100 is a peripherally inserted central catheter having the elongate medical device 105 disposed with the lumen thereof, where the elongate medical device 105 takes the form of a guidewire. The guidewire (or more specifically the optical fiber 106 of the elongate medical device 105) is optically coupled with the shape sensing system 120.

The medical device assembly 100 is inserted into a peripheral vein 210 via a vascular access site 205. The medical device assembly 100 is further advanced along a subclavian vein 211 so that the distal end 101B of the tubular member 101 is disposed with the superior vena cava 212.

The fiber optic gratings 121 (see FIG. 1) enable the shape sensing system 120 to determine a shape of the elongate medical device 105 (i.e., the optical fiber 106) so that the clinician may observe the shape of the elongate medical device 105 in accordance with the location of the elongate medical device 105 within the vasculature during advancement. The magnetic elements 131 (see FIG. 1) enable the magnetic tracking system 130 to track the location of the elongate medical device 105 within the vasculature with respect to the magnetometers 132.

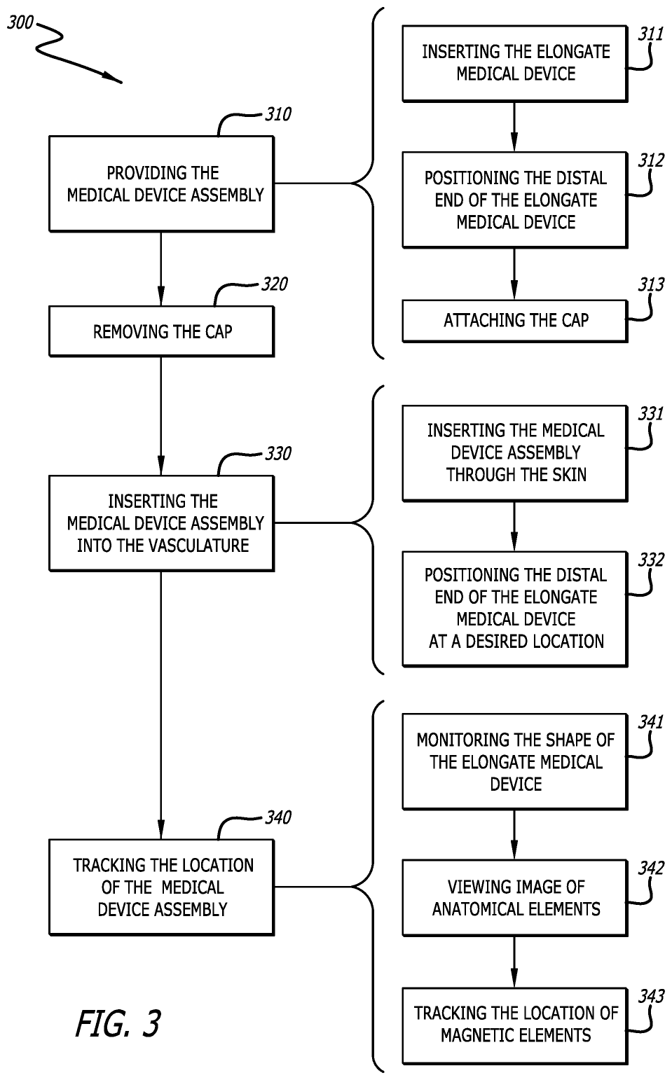

FIG. 3 illustrates a flow chart of a method 300 of inserting the medical device assembly into a patient that may include all or a subset of the following steps or processes. The method 300 may include providing the medical device assembly (block 310). Providing the medical device assembly may, in some embodiments, include inserting the elongate medical device into the lumen of the tubular member (block 311). Inserting the inserting the elongate medical device into the lumen of the tubular member may include positioning the distal end of the elongate medical device at the distal end of the tubular member (block 312). Providing the medical device assembly may further include attaching the cap to the distal end of the tubular member 101 (block 313).

The method 300 may further include removing the cap from the tubular member (block 320).

The method 300 may further include inserting the medical device assembly into the vasculature of the patient (block 330). In some embodiments, inserting the medical device assembly includes removing the cap from the tubular member (block 331). In some embodiments, inserting the medical device assembly includes inserting the distal end of the medical device assembly through a skin of the patient (block 332). In some embodiments, inserting the medical device assembly includes advancing the medical device assembly along the vasculature of the patient (block 333) so that the distal end of the tubular member is positioned at a desired location within the vasculature.

The method may further include tracking the location of the elongate medical device while advancing the medical device assembly (block 340) to ensure that the distal end of the tubular member is positioned at the desired location.

In some embodiments, tracking the location includes monitoring the shape of the elongate medical device via fiber optic shape sensing of the elongate medical device (block 341), which may include viewing the shape of the elongate medical device depicted on a display of a fiber optic shape sensing system.

In some embodiments, tracking the location of the medical device assembly 100 includes viewing an image of anatomical elements adjacent the distal end of the elongate medical device (block 342). Viewing the image may include propagating illuminating light distally along the elongate medical device and projecting the illuminating light away from the distal end of the elongate medical device. In such embodiments, the method may further include propagating imaging light proximally along the elongate medical device in response to the projected illuminating light. In some embodiments, tracking the location of the medical device assembly 100 may include viewing images of anatomic elements on a display, such as the display of the shape sensing system, for example.

In some embodiments, tracking the location includes magnetically tracking the location of the number of magnetic elements with respect to the number of magnetometers of a magnetic tracking system (block 333). In some embodiments, magnetically tracking the location of the number of magnetic elements may include viewing the location of the elongate medical with respect the magnetometers on a display. In some embodiments of the method, tracking the location of the number of magnetic elements includes detecting and tracking the location of the magnetic signature defined by the magnetic elements.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device assembly, comprising:
a tubular member defining a lumen extending along a length of the tubular member from a proximal end to a distal end, the tubular member configured for insertion into a patient;
an elongate medical device disposed within the lumen such that a distal end of the elongate medical device is disposed adjacent the distal end of the tubular member; and
a cap covering the distal end of the tubular member, the cap configured to prevent contamination of the elongate medical prior to insertion of the tubular member into the patient.

2. The assembly according to claim 1, wherein the cap forms a fluid tight seal with the tubular member.

3. The assembly according to claim 1, wherein the cap is configured to prevent microbial ingress into the lumen.

4. The assembly according to claim 1, wherein the cap is detachably coupled with the tubular member.

5. The assembly according to claim 1, wherein the elongate medical device is configured to enable tracking of a location of the elongate medical device within the patient.

6. The assembly according to claim 1, wherein the elongate medical device includes an optical fiber extending along a length of the elongate medical device.

7. The assembly according to claim 6, wherein the optical fiber is multicore optical fiber including a plurality of fiber optic gratings disposed along a length of the optical fiber, the fiber optic gratings configured to enable shape sensing of the elongate medical device.

8. The assembly according to claim 7, wherein the multicore optical fiber includes:
a number of illuminating optical fiber cores configured for propagating illuminating light distally along the elongate medical device and projecting the illuminating light away from the distal end of the elongate medical device, and
a number of imaging optical fiber cores configured for propagating imaging light proximally along the elongate medical device, the imaging received at the distal end of the elongate medical device in response to the illuminating light.

9. The assembly according to claim 1, wherein:
the elongate medical device includes a number of magnetic elements disposed along at least a distal portion of the length of the medical device, and
the magnet elements are configured to enable magnetic tracking of a location the elongate medical device.

10. The assembly according to claim 9, wherein the number of magnetic elements define a magnetic signature for the medical device, the magnetic signature distinct from other magnetic signatures for other medical devices.

11. The assembly according to claim 1, wherein the tubular member is an intravascular catheter.

12. The assembly according to claim 1, wherein the elongate medical device is a stylet, a catheter, a needle, an obturator, or a sheath.

13. A medical cap for a vascular catheter, comprising:
a cap body defining a proximal end and a distal end; and
a cavity extending into the cap body from the proximal end, the cavity configured to receive and couple with the catheter,
wherein the cap is configured prevent contamination of the medical device disposed within a lumen of the catheter.

14. The cap according to claim 13, wherein the cap forms a fluid tight seal with the catheter.

15. The cap according to claim 13, wherein the cap is configured to prevent microbial ingress into the lumen.

16. The cap according to claim 13, wherein the cap is configured to detachably couple with the catheter.

17. The cap according to claim 13, wherein the cavity includes a tapered inlet.

18. The cap according to claim 13, wherein a distal end of the cap includes a bullnose shape.

19. A method of inserting a medical device into a patient, comprising:
providing an elongate medical device assembly, comprising:
a tubular member defining a lumen extending along a length of the tubular member from a proximal end to a distal end, the tubular member configured for insertion into the patient;
an elongate medical device disposed within the lumen such that a distal end of the elongate medical device is disposed adjacent the distal end of the tubular member;
a cap covering the distal end of the tubular member, the cap configured to prevent contamination of the elongate medical prior to insertion of the tubular member into the patient;
removing the cap from the tubular member; and
inserting the distal end of the medical device assembly through a skin of the patient.

20. The method according to claim 19, wherein the cap forms a fluid tight seal with the tubular member.

21. The method according to claim 19, wherein the cap is configured to prevent microbial ingress into the lumen.

22. The method according to claim 19, further comprising advancing the medical device assembly along a vasculature of the patient.

23. The method according to claim 19, further comprising tracking the location of the elongate medical device during advancing the medical device assembly.

24. The method according to claim 23, wherein:
the elongate medical device includes a multicore optical fiber including a plurality of fiber optic gratings disposed along a length of the optical fiber, the fiber optic gratings configured to enable shape sensing of the elongate medical device, and
tracking the location of the elongate medical device includes monitoring a shape of the elongate medical device depicted on a display of a fiber optic shape sensing system.

25. The method according to claim 24, wherein:
the multicore optical fiber includes:
a number of illuminating optical fiber cores configured for propagating illuminating light distally along the elongate medical device and projecting the illuminating light away from the distal end of the elongate medical device, and
a number of imaging optical fiber cores configured for propagating imaging light proximally along the elongate medical device, the imaging light received at the distal end of the elongate medical device in response to the illuminating light, and
the method further includes:
projecting the illuminating light distally away from the distal end of the elongate medical device; and
receiving the imaging light proximally via the distal end of the elongate medical device.

26. The method according to claim 23, wherein:
the elongate medical device includes a number of magnetic elements disposed along at least a distal portion of the elongate medical device, and
tracking the location of the elongate medical device includes tracking the location of the number of magnetic elements with respect to a number of magnetometers of a magnetic tracking system.

27. The method according to claim 26, wherein the number of magnetic elements define a magnetic signature of the elongate medical device.

28. The method according to claim 27, wherein the magnetic signature is distinct from other magnetic signatures of other elongate medical devices.

29. The method according to claim 19, wherein the tubular device is an intravascular catheter or a sheath.

30. The method according to claim 19, wherein the elongate medical device is a stylet, a catheter, a needle, or an obturator.
